(19) United States
(12) Patent Application Publication
(10) Pub. No.: US 2006/0058241 A1
(43) Pub. Date: Mar. 16, 2006

(54) Methods of treating disorders having a component of mercury toxicity

(76) Inventors: Mark R. Geier, Silver Spring, MD (US); David A. Geier, Silver Spring, MD (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/225,623

(22) Filed: Sep. 13, 2005

Related U.S. Application Data
(63) Continuation-in-part of application No. 10/941,887, filed on Sep. 16, 2004.

(51) Int. Cl. A61K 38/09; A61K 31/56

(52) U.S. Cl. 514/15; 514/171

(57) ABSTRACT

The present invention relates to methods of lowering the level of mercury in a subject, methods of lowering the level of mercury in a child diagnosed with autism and methods of assessing the risk of whether a child is susceptible of developing autism.

### RELATED APPLICATION INFORMATION

This application is a continuation-in-part of U.S. application Ser. No. 10/941,887 filed on Sep. 16, 2004, the contents of which are herein incorporated by reference.

### FIELD OF THE INVENTION

The present invention relates to methods of lowering the level of mercury in a subject determined to contain a high level of mercury, methods of lowering the level of mercury in a child diagnosed with autism and methods of assessing the risk of whether a child is susceptible of developing autism.

### BACKGROUND OF THE INVENTION

Mercury toxicity has been reported throughout history. For example, mercury has been found in Egyptian tombs, indicating it was used as early as 1500 BC. In the late 18th century, antisyphilitic agents contained mercury. During the 1800's, the phrase “mad as a hatter” was coined because of the chronic mercury exposure that the fetters faced because mercury was used in hat making. Today, humans are exposed to mercury from a variety of different sources, including dental amalgams, certain industries such as battery, thermometer and barometer manufacturing, ingestion of certain foods such as fish and shellfish, environmental pollution resulting from the use of fossil foods, and from vaccinations containing thimerosal, a mercury-containing preservative.

Mercury can be found in a variety of different forms. Elemental mercury can be found as a liquid or vapor. Organic mercury can be found in three different forms, aryl and short and long chain alkyl compounds. Examples of organic mercury include, but are not limited to, ethylmercury and methylmercury. Inorganic mercury is found mostly in the form of a mercuric salt, such as mercuric chloride. It is known in the art that mercuric chloride binds and forms a complex with testosterone in subjects (See, Cooper et al., “The Crystal Structure and Absolute Configuration of the 2:1 Complex between Tesosterone and Mercuric Chloride,” Acta Crystallogr B., 1968, 15:24(7):935-41).

Mercury toxicity or poisoning can result from vapor inhalation, ingestion, injection, or absorption through the skin. Exposure to any form of mercury on a repeated basis, or even from a single, very high exposure can lead to mercury toxicity or mercury poisoning. There are three main symptoms or mercury toxicity or mercury poisoning:
1. Gum problems. The gums become soft and spongy, the teeth get loose, sores may develop, and there may be increased saliva.
2. Mood and mental changes. People suffering from mercury toxicity or mercury poisoning often have wide swings of mood, becoming irritable, frightened, depressed or excited very quickly for no apparent reason. Such people may become extremely upset at any criticism, lose all self-confidence, and become apathetic. Hallucinations, memory loss and inability to concentrate can occur.
3. Nervous system. The earliest and most frequent symptom is a fine tremor (shaking) of the hand. A tremor may also occur in the tongue and eyelids. Eventually this can progress to trouble balancing and walking.

In addition, there are a number of other symptoms that may be caused by exposure to high levels of mercury and mercury-containing compounds. For example, skin allergies may develop. If this happens, repeated exposure causes rash and itching. Exposure to mercury vapor can cause the lens of the eye to discolor. In addition, some inorganic mercury compounds can cause burns or severe irritation of the skin and eyes on contact. Moreover, some organic mercury compounds (such as methylmercury) are known to cause birth defects in children born of exposed mothers.

A number of diseases are believed to have a mercury toxicity component. These include autism, Alzheimer's disease (See, Pendergrass J C, Haley B E, Vimy M J, Winfield S A, Lorscheider F L, “Mercury vapor inhalation inhibits binding of GTP to tubulin in rat brain: similarity to a molecular lesion in Alzheimer diseased brain,” Neurotoxicology 1997;18:315-24 and Pendergrass J C, Haley B E., “Inhibition of brain tubulin-guanosine 5′-triphosphate interactions by mercury: similarity to observations in Alzheimer's diseased brain,” Met Ions Biol Syst 1997;34:461-78), diabetes (See, Waly M, Olteanu H, Banerjee R, Choi S W, Mason J B, Parker B S, Sukumar S, Shim S, Sharma A, Benzecry J M, Power-Charnitsky V A, Deth R C., “Activation of methionine synthase by insulin-like growth factor-1 and dopamine: a target for neurodevelopmental toxins and thimerosal,” Mol Psychiatry 2004;9:358-70), heart disease (See, Guallar E, Sanz-Gallardo M I, van't Veer P, Bode P, Aro A, Gomez-Aracena J, Kark J D, Riemersma R A, Martin-Moreno J M, Kok F J; “Heavy Metals and Myocardial Infarction Study Group. Mercury, fish oils, and the risk of myocardial infarction,” N Engl J Med 2002;347:1747-54), obesity (See, Kishida K, Kuriyama H, Funahashi T, Shimomura I, Kihara S, Ouchi N, Nishida M, Nishizawa H, Matsuda M, Takahashi M, Hotta K, Nakamura T, Yamashita S, Tochino Y, Matsuzawa Y., “Aquaporin adipose, a putative glycerol channel in adipocytes,” J Biol Chem. Jul. 7, 2000;275(27):20896-902), amyotrophic lateral sclerosis (ALS) (See, Sillevis Smitt P A, de Jong J M., “Animal models of amyotrophic lateral sclerosis and the spinal muscular atrophies,” J Neurol Sci 1989;91:231-58 and Barber T E., “Inorganic mercury intoxication reminiscent of amyotrophic lateral sclerosis,” J Occup Med 1978;20:667-9), asthma (See, Kazantzis G., “The role of hypersensitivity and the immune response in influencing susceptibility to metal toxicity,” Environ Health Perspect 1978;25:111-8) and certain immune disorders (See, Nakagawa R., “Concentration of mercury in hair of diseased people in Japan,” Chemosphere 1995;30:135-40).

With respect to autism, autism is a neurodevelopmental disorder characterized by impairments in social relatedness and communication, repetitive behaviors, abnormal movements, and sensory dysfunction. According to the most recent estimates published by the Centers for Disease Control and Prevention (CDC), it has been reported that approximately 1 in 150 children in the United States suffers from an autistic disorder, and far more males than females suffer from autistic disorders (See, Bertrand J, Mars A, Boyle C, Bove F, Yeargin-Allsopp M, Decoufle P., “Prevalence of autism in a United States population: the Brick Township, New Jersey, investigation,” Pediatrics 2001;108:1155-61, Yeargin-Allsopp M, Rice C, Karapurkar T, Doemberg N, Boyle C, Murphy C., “Prevalence of autism in a US metropolitan area”, JAAM 2003;289:49-55, Gerlai R, Gerlai J., “Autism: a target for pharmacotherapies?” Drug Discov Today 2004;9:366-74, Gerlai R, Gerlai J., “Autism: a large unmet medical need and a complex research problem,” Physiol Behav, 2003;79:461-70, California Department of Developmental Services. “Autistic Spectrum Disorders—Changes in the California Caseload—An Update: 1999 through 2002,” Sacramento, Calif.: State of California, 2003, Blaxill M F, Baskin D S, Spitzer W O., “Commentary: Blaxill, Baskin, and Spitzer on Croen et al. (2002), the changing prevalence of autism in California. J Autism Dev Disord 2003;33:223-6, Blaxill M F. “What's going on? The question of time trends in autism,” Public Health Rep 2004; 119:536-51 and Newschaffer C J, Falb M D, Gurney J G., “National autism prevalence trends from United States special education data,” Pediatrics 2005;115:e277-82). Furthermore, in recent research observing children's communicative, social, affective, repetitive behaviors, and toy play coded from videotapes of the toddlers' first and second birthday parties there are significant numbers of children with regressive autistic disorders that manifest between the ages of 12 and 24 months of age, a temporal period directly post-administration of mercury from thimerosal-containing childhood vaccines in the United States (See, Werner E, Dawson G., “Validation of the phenomenon of autistic regression using home videotapes,” Arch Gen Psychiatry 2005;62:889-95).

Recent studies have reported that exposure to mercury can cause immune, sensory, neurological, motor, and behavioral dysfunctions similar to traits defining or associated with autism, and the similarities extend to neuroanatomy, neurotransmitters, and biochemistry (See, Bernard S, Enayati A, Redwood L, Roger H, Binstock T., “Autism: a novel form of mercury poisoning,” Med Hypotheses, 2001;56:462-71, Bernard S, Enayati A, Roger H, Binstock T, Redwood L. “The role of mercury in the pathogenesis of autism,” Mol Psychiatry 2002;7 Suppl 2:S42-3 and Blaxill M F, Redwood L, Bernard S., “Thimerosal and autism? A plausible hypothesis that should not be dismissed,” Med Hypotheses 2004;62:788-94.).

Thimerosal, a preservative added to many vaccines, has become a major source of mercury among children in the United States who, within their first two years, may have received a quantity of mercury that exceeded Federal Safety Guidelines (See, Redwood L, Bernard S, Brown D., “Predicted mercury concentrations in hair from infant immunizations: cause for concern,” Neurotoxicology 2001;22:691-7 and Ball L K, Ball R, Pratt R D, “An assessment of thimerosal use in childhood vaccines,” Pediatrics 2001;107: 1147-54). According to the CDC recommended immunization schedule in the United States during the 1990's, infants may have been exposed to 12.5 μg of mercury at birth, 62.5 μg of mercury at 2 months, 50 μg of mercury at 4 months, 62.5 μg of mercury at 6 months, and 50 μg of mercury at 18 months, for a total of 237.5 μg of mercury during the first 18 months of life, if all thimerosal-containing vaccines were administered (See, Redwood L, Bernard S, Brown D, “Predicted mercury concentrations in hair from infant immunizations: cause for concern,” Neurotoxicology 2001 ;22:691-7 and Ball L K, Ball R, Pratt R D., “An assessment of thimerosal use in childhood vaccines,” Pediatrics 2001;107:1147-54.).

Redwood et al. (Redwood L, Bernard S, Brown D, “Predicted mercury concentrations in hair from infant immunizations: cause for concern,” Neurotoxicology 2001;22:691-7) have estimated hair mercury concentrations expected to result from the recommended CDC childhood immunization schedule during the 1990s utilizing a one compartment pharmacokinetic model. The authors determined that modeled hair mercury concentrations in infants exposed to vaccinal thimerosal were in excess of the Environmental Protection Agency (EPA)'s safety guidelines of 1 part-per-million (ppm) for up to the first 365 days, with several peak concentrations within this period. The inventors have evaluated doses of mercury from thimerosal-containing childhood vaccines administered in accordance with the recommended CDC childhood immunization schedule during the 1990s in comparison the EPA and the Food and Drug Administration (FDA) safety guidelines for the oral ingestion of methylmercury, a similar compound to ethylmercury. Geier et al., the inventors of the present invention, reported that children received instantaneous doses of mercury from thimerosal-containing childhood vaccines that were many-fold in excess of the Federal Safety Guidelines (See Geier M R, Geier D A., “Thimerosal in childhood vaccines, neurodevelopment disorders, and heart disease in the United States,” J Am Phys Surg 2003;8:6-1 1 and Geier D A, Geier M R., “An assessment of the impact of thimerosal on neurodevelopmental disorders,” Pediatr Rehabil 2003;6:97-102.). In evaluating the dose of mercury children received from thimerosal-containing vaccines in the US, when factoring in significant environmental exposure (i.e., mercury in breast milk), it has been estimated the mercury in thimerosal-containing vaccines represented almost 50% of the total mercury dose infants received (See, Bigham M, Copes R. “Thiomersal in vaccines: balancing the risk of adverse effects with the risk of vaccine-preventable disease,” Drug Saf, 2005;28:89-101.). As a result, it has been determined that some infants receiving 187.5 μg of mercury from thimerosal-containing vaccines during the first sixth months of life from the routine childhood vaccination schedule, in combination with environmental exposure from mercury in breast milk (164 μg of mercury), were exposed to cumulative doses of mercury during the first sixth months of life in excess of the methylmercury safety guidelines established by the EPA, Health Canada, the World Health Organization (WHO), the Agency for Toxic Substances Disease Registry (ATSDR), and the FDA. It was also determined that these same infants (with no additional exposure to mercury from any source) were in excess of the methylmercury guidelines established by the EPA, Health Canada, WHO, and the ATSDR for the entire first year of life.

In evaluating the distribution of mercury within the body following thimerosal-containing vaccine administration to infants, Burbacher et al. have evaluated infant monkeys following injection of doses of mercury comparable to the US dosing schedule (weight- and age-adjusted) (See, Burbacher T M, Shen D D, Liberato N, Grant K S, Cernichiari E, Clarkson T W., “Comparison of blood and brain mercury levels in infant monkeys exposed to methylmercury or vaccines containing Thimerosal,” Environ Health Perspect 2005;1 13:1015-21). These researchers confirmed that thimerosal crosses the blood-brain barrier and results in appreciable mercury content in tissues including the brain (the maximum concentration observed in the brain was approximately 50-parts-per-billion). They determined that the overall half-life of mercury in the brain of the infant monkeys examined was approximately 24 days. In addition, it was determined that the percentage of inorganic mercury in the brains of the thimerosal-treated infant monkeys averaged 16 parts-per-billion following the dosing schedule, and the half-life of this inorganic mercury was found to be very long in the monkey brains (>120 days).

Furthermore, Hornig et al. administered thimerosal to mice, mimicking the United States' routine childhood immunization schedule of the 1990s (weight- and age-adjusted), and observed autistic symptoms in a susceptible mouse strain that included growth delay, reduced locomotion, exaggerated response to novelty, increased brain size, decreased numbers of Purkinje cells, significant abnormalities in brain architecture, affecting areas sub-serving emotion and cognition, and densely packed hyperchromic hippocampal neurons with altered glutamate receptors and transporters (See, Hornig M, Chian D, Lipkin W I., “Neurotoxic effects of postnatal Thimerosal are mouse strain dependent,” Mol Psychiatry 2004;9:833-45). In addition, Digar et al. showed exposure to thimerosal from injection of a single 50 μg of mercury dose at specific prenatal developmental stages in an animal model resulted in significant fetal lethality and teratogenecity compared to controls (See, Digar A, Sensharma G C, Samal S N., “Lethality and teratogenecity of organic mercury (thimerosal) on the chick embryo,” J Anat Soc India 1987;36:153-9).

In a series of molecular studies with neurons it has now been shown that nanomolar (nM) to micromolar (μM) concentrations of thimerosal are capable of inducing neuronal death, neurodegeneration, membrane damage, and DNA damage within hours of exposure (Baskin D S, Ngo H, Didenko V V., “Thimerosal induces DNA breaks, caspase-3 activation, membrane damage, and cell death in cultured human neurons and fibroblasts,” Toxicol Sci, 2003;74:361-8, Parry J M., “An evaluation of the use of in vitro tubulin polymerisation, fungal and wheat assays to detect the activity of potential chemical aneugens,” Mutation Res 1993;287:23-8, Wallin M, Hartely-Asp B., “Effects of potential aneuploidy inducing agents on microtubule assembly in vitro,” Mutation Res 1993;287:17-22, Brunner M, Albertini S, Wurgler F E., “Effects of 10 known or suspected spindle poisons in the in vitro porcine brain tubulin assembly assay,” Mutagenesis 1991;6:65-70, James S J, Slikker W 3rd, Melnyk S, New E, Pogribna M, Jernigan S., “Thimerosal neurotoxicity is associated with glutathione depletion: protection with glutathione precursors,” Neurotoxicology 2005;26:1-8 and Humphrey M L, Cole M P, Pendergrass J C, Kiningham K K., “Mitochondrial mediated thimerosal-induced apoptosis in a human neuroblastoma cell line (SK—N—SH),” Neurotoxicology 2005;26:407-16). Additionally, it has also been shown that nM to μM concentrations of thimerosal are capable of disrupting critical signaling pathways/biochemical events necessary for neurons to undergo normal neuronal development (See, Parran D K, Barker A, Ehrich M., “Effects of Thimerosal on NGF signal transduction and cell death in neuroblastoma cells,” Toxicol Sci 2005;86:132-40, Waly, et al., “Activation of methionine synthase by insulin-like growth factor-1 and dopamine: a target for neurodevelopmental toxins and thimerosal,” Mol Psychiatry 2004;9:358-70 and Mutkus L, Aschner J L, Syversen T, Shanker G, Sonnewald U, Aschner M., “In vitro uptake of glutamate in GLAST- and GLT-1-transfected mutant CHO-K1 Cells is inhibited by the ethylmercury-containing preservative thimerosal,” Biol Trace Elem Res 2005;105:71-86).

Epidemiological studies conducted in the United States have examined the relationship between thimerosal-containing childhood vaccines and autistic and other neurodevelopmental disorders. It has been shown that children receiving thimerosal-containing childhood vaccines were 2- to 8-fold statistically significantly more likely to develop autistic and other neurodevelopmental disorders, depending upon the symptoms and outcomes examined, in comparison to children receiving thimerosal-free childhood vaccines (See, Geier M R, Geier D A., “Thimerosal in childhood vaccines, neurodevelopment disorders, and heart disease in the United States,” J Am Phys Surg 2003;8:6-11, Geier D A, Geier M R., “An assessment of the impact of thimerosal on neurodevelopmental disorders,” Pediatr Rehabil 2003;6:97-102, Geier M R, Geier D A., “Neurodevelopmental disorders after thimerosal-containing vaccines: a brief communication,” Exp Biol Med 2003;228:660-4 and Geier D A, Geier M R, “A comparative evaluation of the effects of MMR immunization and mercury doses from thimerosal-containing childhood vaccines on the population prevalence of autism,” Med Sci Monit 2004;10(3):PI33-9, Geier D A, Geier M R., “Neurodevelopmental disorders following thimerosal-containing childhood immunizations: a follow-up analysis,” Int J Toxicol 2004;23:369-76, Geier D A, Geier M R., “A two-phased population epidemiological study of the safety of thimerosal-containing vaccines: a follow-up analysis,” Med Sci Monit 2005;11(4):CR160-70.).

Several recent studies have clinically evaluated the body-burden of heavy metals present in children with autistics disorders in comparison to normal children. Bradstreet et al. (See, Bradstreet J, Geier D A, Kartzinel J J, Adams J B, Geier M R, “A case-control study of mercury burden in children with autistic spectrum disorders,” J Am Phys Surg 2003;8:76-9) have evaluated urinary heavy metals following three days of oral chelation with meso-2,3-dimercaptosuccinic acid (DMSA) in children with autistic disorders in comparison to a control population. It was determined that autistic children had statistically significantly approximately 6-fold higher urinary mercury concentrations than matched normal controls, whereas other heavy metals were present in similar urinary concentrations in both groups following three days of oral chelation with DMSA. In addition, in this study, urinary mercury concentrations were compared following three days of oral chelation with DMSA in matched vaccinated and unvaccinated normal children. It was observed that there were similar concentrations of urinary mercury in both groups following DMSA treatment. Holmes et al. (See, Holmes A S, Blaxill M F, Haley B E., “Reduced levels of mercury in first baby haircuts of autistic children,” Int J Toxic 2003;22:277-85) have evaluated first baby haircuts from autistic children in comparison to controls. It was observed that the mercury levels in the first baby haircuts of children were inversely related to the severity of the autistic disorders of the children (i.e. the more severely affected the children are, the less mercury levels were present in their first baby haircuts). It has been hypothesized that these results are consistent with autistic children having biochemical differences than normal children, possibly as a result of genetic polymorphisms, resulting in children with autistic disorders having an increased body-burden of mercury in comparison to normal children.

James et al. (See, James S J, Culter P, Melnyk S, et al., “Metabolic biomarkers of increased oxidative stress and impaired methylation capacity in children with autism” Am J Clin Nutr 2004;80:1611-7) have evaluated the methionine cycle and transsulfuration metabolites in autistic children in comparison age- and sex-matched control children. It was determined that there were significant decreases in the plasma concentration of cysteine (19% reduction) and total glutathione (46% reduction), both of which are crucial for mercury excretion, in autistic children in comparison to control children. Additionally, consistent with the DMSA treatment and first baby haircut study results, it was determined that autistic children had significantly increased oxidative stress (3-fold decrease in total glutathione/oxidized glutathione redox ratio) in comparison to control children.

Boris et al. (See, Boris M, et al., “Association of 5,10-Methylenetetrathydrofolate reductase (MTHFR) gene polymorphisms with autistic spectrum disorders,” J Am Phys Surg 2004;9:106-8 recently conducted genomic studies of children with autistic disorders in comparison to normal control populations. The authors examined genes in pathways that are responsible for the synthesis of key biochemical molecules that are of functional relevance in the excretion and/or oxidative stress protection of mercury from the body. Notably, only 2% of children with autistic disorders examined by the authors did present with at least one single nucleotide polymorphism (SNP) in the MTHFR gene. Additionally, the authors demonstrated that there was approximately a 2-fold statistically significant increase in both the homozygous (677 TT) and heterozygous (677 CT and 1298 AC) SNPs in the MTHFR gene in autistics in comparison to controls. This is of particular relevance because MTHFR is one of the key genes in the biochemical pathway involved with the synthesis of glutathione, a key molecule in the body's natural defenses against mercury, and those with homozygous (677 TT) or heterozygous (677 CT and 1298 AC) SNPs in the MTHFR gene have been found to have an enzyme that functions approximately 50-60% less than those with the wild-type MTHFR gene.

The understanding of the cause of the epidemic has allowed for the design of treatment modalities that address the mercury toxic component of these disorders. These therapies include methods to remove the mercury by such techniques as the use of chelating agents and by corrections in various biochemical pathways that lead to sulphydral-containing compounds that the body uses to rid itself of the mercury (See, Johnson S., “Micronutrient accumulation and depletion in schizophrenia, epilepsy, autism and Parkinson's disease?” Med Hypotheses 2001;56:641-5.).

Clarkson et al. (See, Clarkson T W, Nordberg G F, Sager P R. Reproductive and developmental toxicity of metals. Scand J Work Environ Health. 1985; 11:145-54) have developed a mouse model to evaluate the neurotoxic effects of alkyl mercury exposure on different sexes. The authors reported that two-day-old mice were administered alkyl mercury at 4 mg of mercury/kg/bodyweight (low dose), 8 mg of mercury/kg/bodyweight (high dose), or nor mercury. Animals were sacrificed 24 hours later, and matched sections of brain were prepared. The total number of mitotic figures in the external granule layer of the cerebellar cortex were recorded and classified as early (prophase and metaphase) or late (anaphase and telophase). Mercury concentrations in the brain for both males and females were 2.7 micrograms of mercury/gram at the high dose exposure and 1.8 micrograms of mercury at the low dose exposure. The authors determined that at the high dose, male and female mice had similarly reduced percentages of late mitotic figures compared with controls. At the lower dose, female mice were significantly much less affected in their percentages of late mitotic figures compared with male mice. The authors concluded males are considerably more sensitive to the neurotoxic effects of mercury, and that in some human fetal/infant population exposures to low dose alkyl mercury, it has been observed that males were more sensitive than females to psychomotor retardation (See, Clarkson T W, Nordberg G F, Sager P R., “Reproductive and developmental toxicity of metals,” Scand J Work Environ Health. 1985;11:145-54 and Grandjean P, Weihe P, White R F, Debes F., “Cognitive performance of children prenatally exposed to “safe” levels of methylmercury,” Environ Res 1998;77:165-72) Muraoka and Itoh (Muraoka Y, Itoh F., “Sex difference of mercuric chloride-induced renal tubular necrosis in rats—from the aspect of sex differences in renal mercury concentration and sulfhydryl levels—,” J Toxicol Sci 1980;5:203-14) have investigated sex differences in the effects of mercury exposure on other organ systems. The authors reported that when doses of 0.3 to 2 mg/kg of mercuric chloride were intravenously administered to rats of the JCL-SD strain, acute renal tubular necrosis was produced in the straight portion of the proximal tubules with a pronounced sex difference, the male being more susceptible. Necrosis was inhibited by castration of male rats and promoted by testosterone pretreatment.

Researchers (See, Manning J T, Baron-Cohen S, Wheelwright S, Sanders G., “The 2nd to 4th digit ratio and autism,” Dev Med Child Neurol 2001;43:160-4 and Lutchmaya S, Baron-Cohen S, Raggatt P, Knickmeyer R, Manning J T., “2nd to 4^{th} digit ratios, fetal testosterone and estradiol,” Early Hum Dev 2004;77:23-8) have investigated prenatal testosterone levels in children with autistic spectrum disorders. The authors examined 72 children with autism, including 23 children with Aspergers syndrome (i.e. these children have less serve autistic affects), 34 siblings, 88 fathers, 88 mothers, and sex and age-matched controls. The authors demonstrated that the more severely affected the children were the higher the levels of prenatal testosterone.

Currently, there is a need in the art for new methods of treating subjects diagnosed with a high level of mercury and who suffer from mercury poisoning. In addition, there is also a need in the art for methods of treating subjects diagnosed with diseases or disorders that have a mercury component. The present invention discloses new ways to screen and treat disorders that have a mercury component by utilizing the interaction of mercurials with the steroidgenesis pathway and its control by the hypothalamus-pituitary-adrenal axis.

### SUMMARY OF THE PRESENT INVENTION

In one embodiment, the present invention relates to a method of lowering the level of mercury in a subject diagnosed or suffering from mercury toxicity. The method comprises the following steps:

a) administering to said subject a pharmaceutically effective amount of at least one luteinizing hormone releasing hormone composition;

b) administering to said subject a pharmaceutically effective amount of at least one chelating agent; and

c) repeating step a) or step b) or steps a) and b) as necessary to lower the level of mercury in said subject.

The method can also further optionally comprise the step of administering to the subject a pharmaceutically effective amount of at least one antiandrogenic hormone either prior to or after step c).

The at least one luteinizing hormone composition used in the above-described method can be a luteinizing hormone releasing hormone (“LHRH”) analogue, a LHRH agonist, a LHRH antagonist or combinations thereof. The at least one chelating agent administered pursuant to the above-described method can be administered orally, transdermally, intravenously, orally and transdermally, orally, transdermally and intravenously, orally and intravenously or transdermally and intravenously. The at least one antiandrogenic hormone, if used in the above-described method, can be cyproterone acetate, finasteride, bicalutamide, novaldex, nilandron, flutamide, progesterone or combinations thereof.

The subject treated pursuant to the above-described method can be a human male or female, adult or child. If the subject being treated is a child, said child can have an age between two (2) years old and seventeen (17) years old. In addition, the human male or human female subject may also be suffering from a disorder selected from the group consisting of: autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, migraine, toxic encephalopathy, cerebral degenerations, anterior horn cell disease, spinocerebellar disease, extrapyramidal disease or myopathy. Preferably, the subject is a male child who has autism. More preferably, the subject is a male child, who has autism and who has also been diagnosed with precocious puberty.

In another embodiment, the present invention relates to a method of lowering the level of mercury in a human child suffering from mercury toxicity, wherein said child also suffers from autism. The method involves the following steps:

a) administering to said child a pharmaceutically effective amount of at least one luteinizing hormone releasing hormone composition;

b) administering to said child a pharmaceutically effective amount of at least one chelating agent; and

c) repeating step a) or step b) or steps a) and b) as necessary to lower the level of mercury in said subject.

The method can also further optionally comprise the step of administering to the subject a pharmaceutically effective amount of at least one antiandrogenic hormone either prior to or after step c).

The at least one luteinizing hormone composition used in the above described method can be a luteinizing hormone releasing hormone (“LHRH”) analogue, a LHRH agonist, a LHRH antagonist or combinations thereof. The at least one chelating agent administered pursuant to the above-described method can be administered orally, transdermally, intravenously, orally and transdermally, orally, transdermally and intravenously, orally and intravenously or transdermally and intravenously. The at least one antiandrogenic hormone, if used in the above described method, can be cyproterone acetate, finasteride, bicalutamide, novaldex, nilandron, flutamide, progesterone or combinations thereof.

The subject treated pursuant to the above-described method can be a human child, male or female. The child treated pursuant to this method can have an age between two (2) years old and seventeen (17) years old. Preferably, the subject is a male child, who has autism and who has also been diagnosed with precocious puberty.

In a third embodiment, the present invention relates to a method of treating a child diagnosed with autism, wherein said child is also diagnosed with mercury toxicity. The method comprises the steps of:

a) administering to said child a pharmaceutically effective amount of at least one luteinizing hormone releasing hormone composition;

b) administering to said child a pharmaceutically effective amount of at least one chelating agent; and

c) repeating step a) or step b) or steps a) and b) as necessary to treat said child.

The method can also further optionally comprise the step of administering to the subject a pharmaceutically effective amount of at least one antiandrogenic hormone either prior to or after step c).

The at least one luteinizing hormone composition used in the above described method can be a luteinizing hormone releasing hormone (“LHRH”) analogue, a LHRH agonist, a LHRH antagonist or combinations thereof. The at least one chelating agent administered pursuant to the above-described method can be administered orally, transdermally, intravenously, orally and transdermally, orally, transdermally and intravenously, orally and intravenously or transdermally and intravenously. The at least one antiandrogenic hormone, if used in the above described method, can be cyproterone acetate, finasteride, bicalutamide, novaldex, nilandron, flutamide, progesterone or combinations thereof.

The subject treated pursuant to the above-described method can be a human child, male or female. The child treated pursuant to this method can have an age between two (2) years old and seventeen (17) years old. Preferably, the subject is a male child, who has autism and who has also been diagnosed with precocious puberty.

In a fourth embodiment, the present invention relates to a method of assessing the risk of whether a child is susceptible of developing autism. The method involves the following steps:

a) determining the level of total serum testosterone from a test sample obtained from a child; and

b) assessing, based on a comparison of the level of total serum testosterone in said test sample with a reference level of total serum testosterone, whether said child is at risk of developing autism.

In the above-described method, a child is at risk of developing autism when said child has a total serum testosterone level that is at the reference level or greater than the reference level for total serum testosterone for a child of approximately the same age. In contrast, a child is not at risk of developing autism when said child has a total serum testosterone level that is lower than the reference level for total serum testosterone for a child of approximately the same age.

### BRIEF DESCRIPTION OF THE DRAWINGS

FIG. 1 shows a description of the precursors to testosterone and estrogen in the steroidgenic pathway.

FIG. 2 shows the role of mercury and glutathione in the testosterone pathway.

FIG. 3 shows the breakdown pathway for testosterone.

### DETAILED DESCRIPTION OF THE INVENTION

### DEFINITIONS

The terms “administer”, “administering”, “administered” or “administration” refer to any manner of providing a drug or pharmaceutically active agent (such as, at least one luteinizing hormone releasing hormone composition, at least one chelating agent, at least one antiandrogenic hormone, etc.) to a subject or patient. Routes of administration can be accomplished through any means known by those skilled in the art. Such means include, but are not limited to, oral, buccal, intravenous, subcutaneous, intramuscular, by inhalation, transdermal and the like.

As used herein, the term “antiandrogenic hormone” refers to any pharmaceutically acceptable active agent that inhibits competitively the effect of androgens at their target site of action. Examples of antiandrogenic hormones that can be used in the present invention include, but are not limited to, cyproterone acetate, finasteride, bicalutamide, novaldex, nilandron, flutamide, progesterone or combinations thereof.

As used herein, the term “chelating agent” refers to any pharmaceutically active agent that is capable of binding or bonding to a mineral or metal present in a subject and then carrying that mineral or metal through the bloodstream to be excreted in the urine of said subject. Chelating agents can be administered to a subject orally, intravenously, subcutaneously, intramuscularly, transdermally, etc. Examples of chelating agents that can be used in the present invention include, but are not limited to, ethylenediaminetetraacetic acid (EDTA), DMSA, sodium dimercaptopropanesulfonate (DMPS), monoisoamyl DMSA (MiADMSA), etc.

By an “effective amount” or a “pharmaceutically effective amount” of a drug or pharmaceutically active agent, such as, at least one luteinizing hormone releasing hormone composition, at least one chelating agent, at least one antiandrogenic hormone, etc., is meant a nontoxic but sufficient amount of the drug or pharmaceutically active agent to provide the desired effect. The amount of drug or pharmaceutically active agent that is “effective” will vary from subject to subject, depending on the age and general condition of the individual, the particular drug or pharmaceutically active agent and the like. Thus, it is not always possible to specify an exact “effective amount.” However, an appropriate “effective amount” in any individual case may be determined by one of ordinary skill in the art using routine experimentation.

The term “gonadotropin” or “gonadotropins” refers to protein hormones secreted by gonadotrope cells of the pituitary gland of mammals. The two principal gonadotropins are luteinizing hormone (“LH”) and follicle stimulating hormone (“FSH”).

The term “luteinizing hormone releasing hormone” (also known as “gonadotropin-releasing hormone” or “GNRH”) or “LHRH” refers to hormone that is a decapeptide having the following structure:
(Pyr)-Glu-His-Trp-Ser-Tyr-Gly-Leu-Arg-Pro-Gly-NH₂

The term “luteinizing hormone releasing hormone composition” or “LHRH composition” refers to a LHRH (or GNRH) analogue, a LHRH (or GNRH) agonist, a LHRH (or GNRH) antagonist or any combination of a LHRH analogue, LHRH agonist or LHRH antagonist that is capable of binding to the LHRH receptor. Preferably, the LHRH analogue, LHRH agonist, LHRH antagonist or combination of LHRH analogue, LHRH agonist or LHRH antagonist is capable of binding to one or more LHRH receptors and are gonadotropin secretory inhibitors or gonadotropin receptor effect blockers.

LHRH agonists that can be used in the present invention can, for example, include the peptides described in Treatment with LHRH analogs: Controversies and perspectives, The Parthenon Publishing Group Ltd. (1996), JP-A-3-503165, JP-A-3-101695, JP-A-7-97334 and JP-A-8-259460 and the like. More specifically, a peptide having the formula:
(Pyr)Glu-R¹-Trp-Ser-R²—R³—R⁴-Arg-Pro-R⁵ (I)

wherein R¹ is His, Tyr, Trp or p-NH₂-Phe; R² is Tyr or Phe; R³ is Gly or D type amino acid residue that may optionally have one or more substituents; R⁴ is Leu, Ile or Nle; and R⁵ is Gly-NH—R⁶ (R⁶ is a hydrogen atom or an alkyl group optionally having a hydroxyl group), NH—R⁷ (R⁷ is a hydrogen atom, an amino group, an alkyl group optionally having a hydroxyl group, or an ureido group (—NH—CO—NH₂)), or a salt thereof, can be used in the present invention.

In the aforementioned formula (I), when R³ is a D type amino acid residue, said D type amino acid can be an α-D-amino acid having up to 9 carbon atoms (i.e., D-Leu, Ile, Nle, Val, Nval, Abu, Phe, Phg, Ser, Thr, Met, Ala, Trp, α-Aibu) or the like. Examples of the substitutents that can be used with R³, include, but are not limited to, tert-butyl, tert-butoxy, tert-butoxycarbonyl, methyl, dimethyl, trimethyl, 2-naphthyl, indolyl-3-yl, 2-methylindolyl, benzyl-imidazo-2-yl and the like. Additionally in formula (I), examples of an alkyl group for R⁶ or R⁷, include, but are not limited to, a C₁₋₄ alkyl group, which is exemplified by methyl, ethyl, propyl, isopropyl, butyl, isobutyl, sec-butyl and tert-butyl.

In addition, a salt of the peptide represented by the formula (I) (which is also referred to as “peptide (I)” herein), include, but are not limited to, an acid salt (i.e., carbonate, bicarbonate, acetate, trifluoroacetate, propionate, succinate etc.) and a metal complex compound (i.e., copper complex, zinc complex etc.) are used. Peptide (I) or a salt thereof can be produced using any method known to those skilled in the art, such as a method described in, for example, U.S. Pat. Nos. 3,853,837, 4,008,209, 3,972,859, GB patent No. 1,423,083, Proceedings of the National Academy of Sciences of the United States of America, vol. 78, pp. 6509-6512 (1981) and the like or a method analogous thereto.

Preferably, peptide (I) can be any one of the following having the below described formulas (a)-(j).

(a) Leuprorelin, a peptide of the formula (I), wherein R¹=His, R²=Tyr, R³=D-Leu, R⁴=Leu, R⁵═NHCH₂—CH₃; 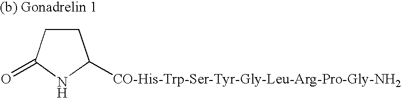
(See, DE Patent No. 2213737); 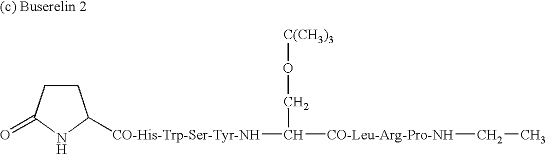
(See, U.S. Pat. No. 4,024,248, DE patent No. 2438352, JP-A-51-41359); 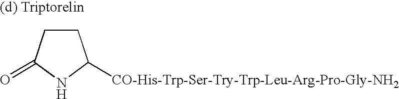
(See, U.S. Pat. No. 4,010,125 and JP-A-52-31073); 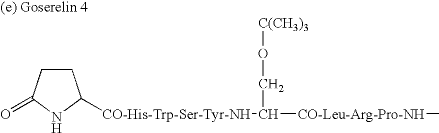
(See, U.S. Pat. No. 4,100,274 and JP-A-52-136172); 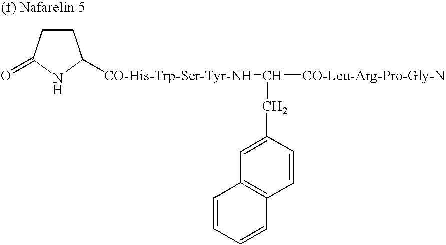
(See, U.S. Pat. No. 4,234,571, JP-A-55-164663, JP-A-63-264498 and JP-A-64-25794); 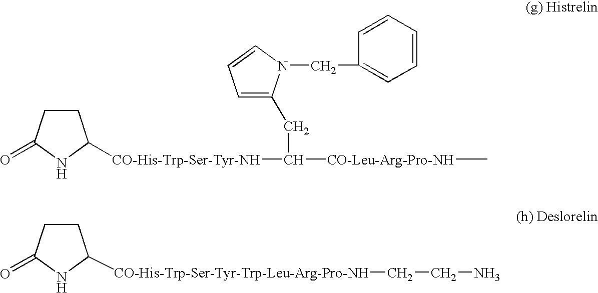
(See, U.S. Pat. No. 4,569,967 and U.S. Pat. No. 4,218,439); 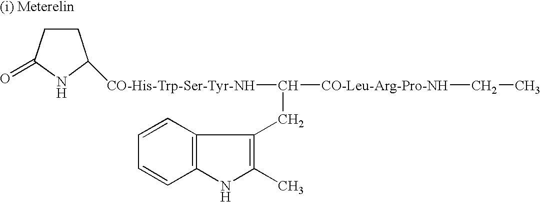
(See, WO 9118016); 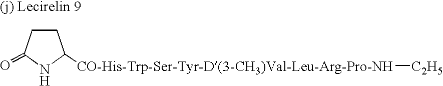
(See Belgium patent No. 897455 and JP-A-59-59654) and the like.

In the aforementioned formulas (c)-(j), an amino acid corresponding to R³ in the formula (I) is in a D-form. The peptide (I) or a salt thereof is preferably leuprorelin or leuprorelin acetate. As used herein, the term “leuprorelin acetate” refers to an acetate of leuprorelin.

LHRH antagonists that can be used in the present invention can, for example, include those disclosed in U.S. Pat. Nos. 4,086,219, 4,124,577, 4,253,997 and 4,317,815, or a peptide represented by the following formula:

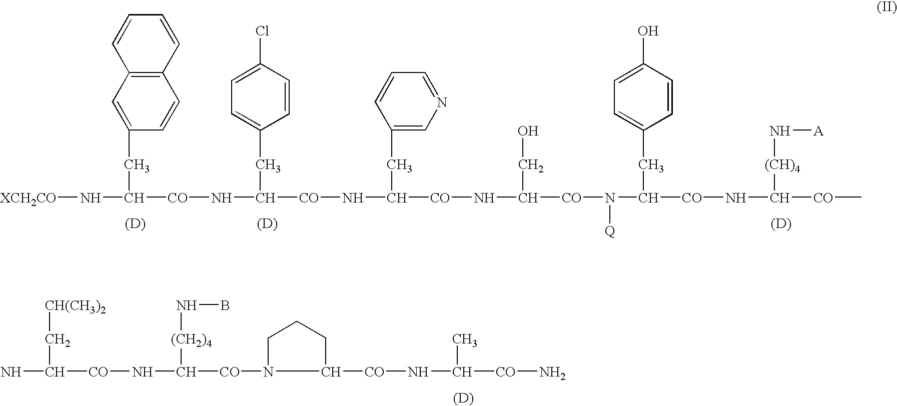

wherein X is a hydrogen or tetrahydrofurylcarboxamide, Q is a hydrogen or methyl, A is nicotinoyl or N,N′-diethylamidino and B is isopropyl or N,N′-diethylamidino (hereinafter is also referred to as “peptide (II)” herein) or a salt thereof. In formula (II), X is preferably tetrahydrofurylcarboxamide, more preferably (2S)-tetrahydrofurylcarboxamide. A is preferably nicotinoyl. B is preferably isopropyl. When peptide (II) has one or more kinds of asymmetric carbon atoms, two or more kinds of optical isomers can be present. Peptide (II) can be used as such optical isomer, or a mixture of these optical isomers.

With respect to a salt of peptide (II), a pharmacologically acceptable salt is preferably used. Examples of such salts, include, but are not limited to, salts of inorganic acids (i.e., hydrochloric acid, sulfuric acid, nitric acid and the like), salts of organic acids (i.e., carbonic acid, bicarbonic acid, succinic acid, acetic acid, propionic acid, trifluoroacetic acid and the like) and the like. Preferably, the salt of peptide (II) is a salt of an organic acid (i.e., carbonic acid, bicarbonic acid, succinic acid, acetic acid, propionic acid, trifluoroacetic acid and the like). Most preferably, the salt of peptide (II) is a salt of acetic acid. More specifically, these salts can be mono, di or tri salts.

More specifically, the peptide (II) or a salt thereof preferably has the following formulas (1)-(4):

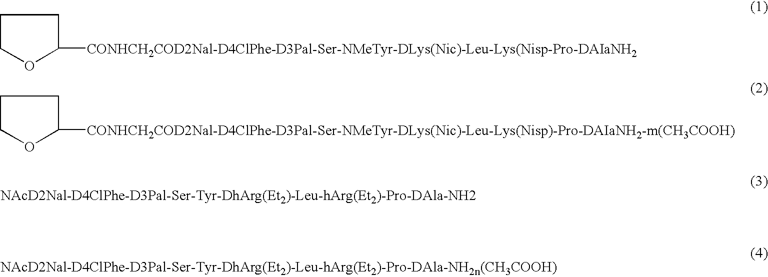

where m is a number of from 1 to 3 and n is a number of from 1 to 3.

The aforementioned formulas (2) and (4) show salts or solvates. Preferably, peptide (II) or a salt thereof has the aforementioned formula (1) or (2), which is particularly preferably an S-isomer.

Peptide (II) or a salt thereof can be produced by any method known to those skilled in the art, such as a method described in JP-A-3-101695 (EP-A 413209), Journal of Medicinal Chemistry, Vol. 35, p. 3942 (1992) and the like, or a method analogous thereto.

Additionally, it is possible to use a linear peptide which is a derivative of LHRH (U.S. Pat. No. 5,140,009 and U.S. Pat. No. 5,171,835), a cyclic hexapeptide derivative (JP-A-61-191698), a bicyclic peptide derivative (Journal of Medicinal Chemistry, Vol. 36, pp. 3265-3273 (1993)) and the like. Examples of non-peptide compounds having an LHRH antagonistic action, compounds described in JP-A-62-116514, WO 95/28405 (JP-A-8-295693), WO 97/14697 (JP-A-9-169767), WO 97/14682 (JP-A-9-169735), WO 96/24597 (JP-A-9-169768), J. Med. Chem., Vol. 32, pp. 2036-2038 (1989) and the like can be used.

Examples of LHRH antagonists that can be used in the present invention include, but are not limited to, abarelix, ganirelix, cetrorelix, 5-(N-benzyl-N-methylaminomethyl)-1-(2,6-difluorobenzyl)-6-[4-(3-methoxyureido)phenyl]-3-phenylthieno[2,3-d]pyrimidine-2,4(1H,3H)-dione, 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione and 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione hydrochloride.

As used herein, the term “precocious puberty” refers to the appearance of physical and hormonal signs of puberty at an earlier age in a subject, preferably a human, than is considered normal. In human girls, precocious puberty is when any of the following develop before eight (8) years of age: breasts, armpit or pubic hair, a rapid height growth (or “growth spurt”), acne, mature external genitalia and/or first menstruation. In human boys, precocious puberty is when any of the following develop before nine (9) years of age: enlarge testes and penis, armpit or pubic hair, a rapid height growth (or “growth spurt”), voice deepening, acne and/or facial hair. Sex steroid levels can be used to determine and diagnose whether a child is suffering from precocious puberty. For example, in boys, total serum testosterone levels can be examined. Methods for determining total serum testosterone levels are known in the art (such as taking a whole blood sample from a subject). Typically, total serum testosterone levels are usually determined in ng/dL. Depending on the reference level used by a laboratory, total serum testosterone levels of 10-30 ng/dL can represent a high level of testosterone that is associated with early or precocious puberty. In girls, estradiol levels can be used to determine and diagnose whether a child is suffering from precocious puberty. Methods for determining estradiol levels are well known in the art (such as taking a whole blood sample from a subject). Estradiol levels are usually determined in pg/mL. Depending on the reference level used by a laboratory, estradiol levels exceeding 20 pg/mL usually represent a high level of estradiol that is associated with early or precocious puberty.

As used herein, the term “subject” refers to an animal, preferably a mammal, including a human or non-human. The terms patient and subject may be used interchangeably herein.

As used herein, the term “test sample” refers to a biological sample obtained from a subject of interest. For example, a test sample can be a biological fluid (e.g., whole blood, serum, plasma, spinal fluid, urine, etc.), a cell sample, or tissue, feces, hair, etc.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

The Present Invention

In one embodiment, the present invention relates to methods for lowering the level of mercury in a subject. More specifically, the methods of the present invention can be used to lower the level of mercury in a subject that has been determined to have a high level of mercury and is diagnosed or suffering from mercury toxicity. Any medical test known to those skilled in the art can be used to determine the level of mercury in a test sample obtained from a subject. The specific type of medical test performed on the test sample is not critical provided that it is capable of determining the level of mercury in a test sample obtained from said subject.

A number of medical tests are known to those skilled in the art for measuring the level of mercury in a subject, particularly a human. For example, one medical test that can be used measures the level of mercury in the whole blood of a subject. Such whole blood tests are well known to those skilled in the art. These blood tests can measure exposure to all three types of mercury (namely, elemental, organic and inorganic mercury). However, because mercury remains in the bloodstream for only a few days after exposure, such a blood test must be done soon after exposure. Typically, non-exposed subjects have mercury levels of 0 to 2 micrograms of mercury per deciliter of blood (μg/dl). Nonetheless, any subject who has a mercury level in his or her whole that is above a laboratory reference level for mercury is considered to have a “high level of mercury” and thus suffering from mercury toxicity for the purposes of this invention.

Another medical test that can be used to measure the level of mercury in a test sample obtained subject is a urine test. Such urine tests are well known to those skilled in the art. Some urine tests measure mercury levels in Vtg/L and some urine tests measure mercury levels in μg/g creatinine. However, the units used to measure the levels of mercury in a subject are not critical. Regardless of which unit of measurement is used, these urine tests measure exposure to elemental and inorganic mercury. Organic mercury cannot be measured as it is not passed out of the body via urine but rather via the feces. Typically, with urine tests that measure mercury levels in μg/L, non-exposed subjects, who do not suffer from mercury toxicity, frequently have urine mercury levels of 0 to 20 μg/L. However, with urine tests that measure mercury levels in μg/g creatinine, subjects that do not suffer from mercury toxicity, have urine mercury levels less than 3.0 μg/g creatinine. Nonetheless, any subject who has a urine mercury level that is at or above a laboratory reference level for mercury is considered to have a “high level of mercury” and thus suffering from mercury toxicity for the purposes of this invention. Additionally, not only are these urine tests used to determine the levels of mercury in a subject, but these tests can also be used to gauge the efficacy of chelation therapy in a subject.

In the present invention, for example, a whole blood test, a urine test or a combination of a whole blood test or a urine test can be used to determine the level of mercury in a subject. Based on the results of the medical test, a determination is made by one skilled in the art whether the level of mercury in said subject is high and whether said subject is suffering from mercury toxicity.

Once a determination has been made that a subject has a high level of mercury and is likely suffering from mercury toxicity, the subject can be treated pursuant to the methods of the present invention in order to lower the level of mercury in said subject. More specifically, the methods of the present invention involve administering to said subject a pharmaceutically effective amount of at least one luteinizing hormone releasing hormone composition. Preferably, the at least one luteinizing hormone releasing hormone is a LHRH agonist, such as leuprolide acetate. For example, leuprolide acetate is available as LUPRON® and LUPRON DEPOT® (Takeda Pharmaceutical Company Limited, Osaka, Japan). LUPRON DEPOT® is available in adult does of 3.75 mg, 7.5 mg, 11 mg, 11.25 mg, 22.5 and 30 mg and in pediatric doses of 7.5 mg, 11.25 mg and 15 mg dosage forms. LUPRON® is available in adult and pediatric daily doses of 5 mg/ml in 2.8 ml multi-dose vials. The methods of the present invention involve administering to said subject at least one luteinizing hormone releasing hormone as a LHRH agonist, such as LUPRON®, either in daily doses of from about 20 ug/kg per day to about 150 ug/kg per day for children (ages 18 years or younger) or about 0.3 to about 5 mg per day to adults or about 2.5 mg to about 100 mg via LUPRON DEPOT® injection for adults or about 5 mg to about 100 mg LUPRON DEPOT® injection for children.

In addition to the pharmaceutically effective amount of at least one luteinizing hormone releasing hormone composition, the subject is also administered a pharmaceutically effective amount of at least one chelating agent. For the purposes of the present invention, the at least one luteinizing hormone releasing hormone composition can be administered first to the subject followed by the pharmaceutically effective amount of at least one chelating agent (one the same day or on a different day), or the pharmaceutically effective amount of at least one chelating agent can be administered first to the subject followed by a pharmaceutically effective amount of at least one luteinizing hormone releasing hormone (on the same day or on a different day).

Any pharmaceutically acceptable chelating agent can be used. As alluded to above, the chelating agent can be administered to the subject on the same day that the subject is administered the at least one luteinizing hormone releasing hormone composition or the at least one chelating agent can be administered on a different day when the subject is not receiving the at least one luteinizing hormone releasing composition. However, once treatment with a pharmaceutically effective amount of at least one chelating agent treatment has been begun in a subject, administration of the pharmaceutically effective amount at least one chelating agent or treatment to the subject or treatment with the pharmaceutically effective amount at least one chelating agent is continued every day (once or multiple times a day), every other day (once or multiple times a day) or every few days (once or multiple times a day) as needed (i.e., until the level of mercury in the subject has been lowered). In addition, the methods of the present invention contemplate treating a subject with a pharmaceutically effective amount of more than one chelating agent at a time, preferably, as different dosage forms. For example, the present invention contemplates treating a subject with a pharmaceutically effective amount of at least one chelating agent (i.e., a first chelating agent) that is administered transdermally as well as with a pharmaceutically effective amount of at least one chelating agent (i.e., a second chelating agent) that is to be administered orally. Each of these chelating agents (i.e., the first and second chelating agents) can be administered separately, on different days, or on the same day. The treatment with each of these chelating agents (i.e., the first and second chelating agents) can be separate from one another (i.e., the first chelating agent is administered for a period of time and then stopped and treatment with the second chelating agent is begun immediately thereafter), overlap with one another (i.e., the first chelating agent is administered for a period of time and then stopped, but prior to stopping treatment with the first chelating agent, treatment with the second chelating agent is begun), or occur concurrently with one another (i.e., the first and second chelating agents are administered at the same time) and with the administration of the at least one luteinizing releasing hormone composition. The amount of at least one chelating agent to be administered to a subject will vary depending on the chelating agent used and how the chelating agent is to be administered (i.e., such as orally, transdermally, intravenously, orally and transdermally, orally, transdermally and intravenously, orally and intravenously or transdermally and intravenously, etc.). Those skilled in the art will be able to determine the type of chelating agent and amount to be given to a subject. For example, oral DMSA can be given to a child at a dose of from about 2 to about 15 mg/kg and such a dose can be administered to said child three times per day. In contrast, transdermal DMPS can be given to a child by applying from about 0.5 to about 5 mg/kg once a day.

Optionally and if necessary, the subject can also be administered a pharmaceutically effective amount of at least one antiandrogenic hormone. Any pharmaceutically acceptable antiandrogenic hormone can be used in the methods of the present invention. The amount of at least one antiandrogenic hormone to be administered to a subject can be from about 50 to about 500 mg per day. The at least one antiandrogenic hormone can be administered to the subject on the same day that the subject is administered the at least one luteinizing hormone releasing hormone composition, at least one chelating agent, at least one luteinizing hormone releasing hormone composition and at least one chelating agent, or on a different day. Additionally, once treatment has begun with the at least one antiandrogenic hormone in a subject, the at least one antiandrogenic hormone can continued to be administered to the subject every day (once or multiple times a day), every other day (once or multiple times a day) or every few days (once or multiple times a day) as needed (i.e., until the level of mercury in the subject has been lowered).

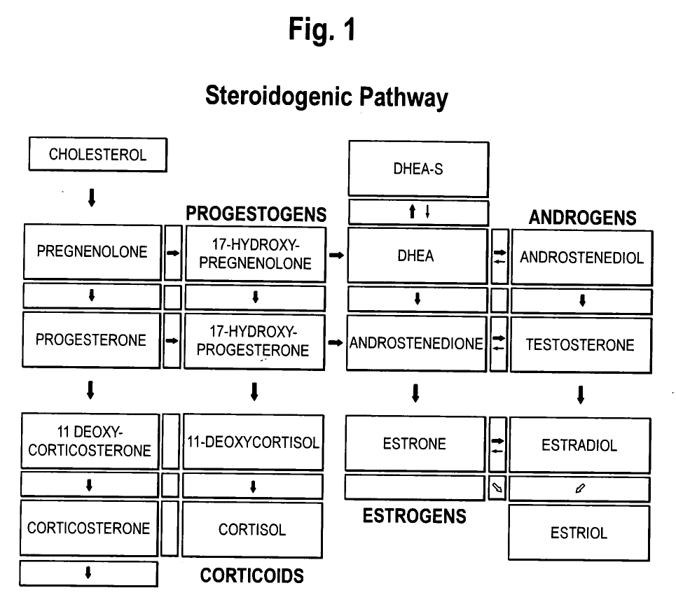

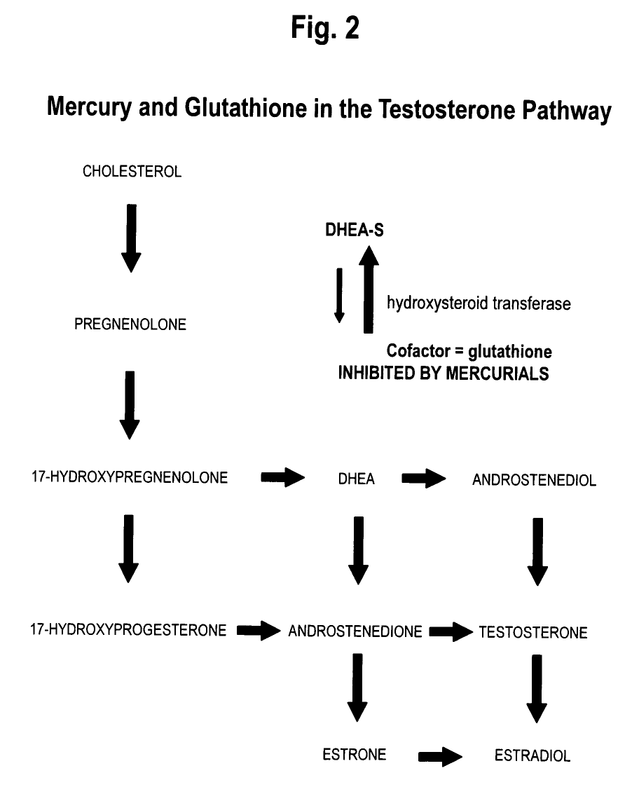

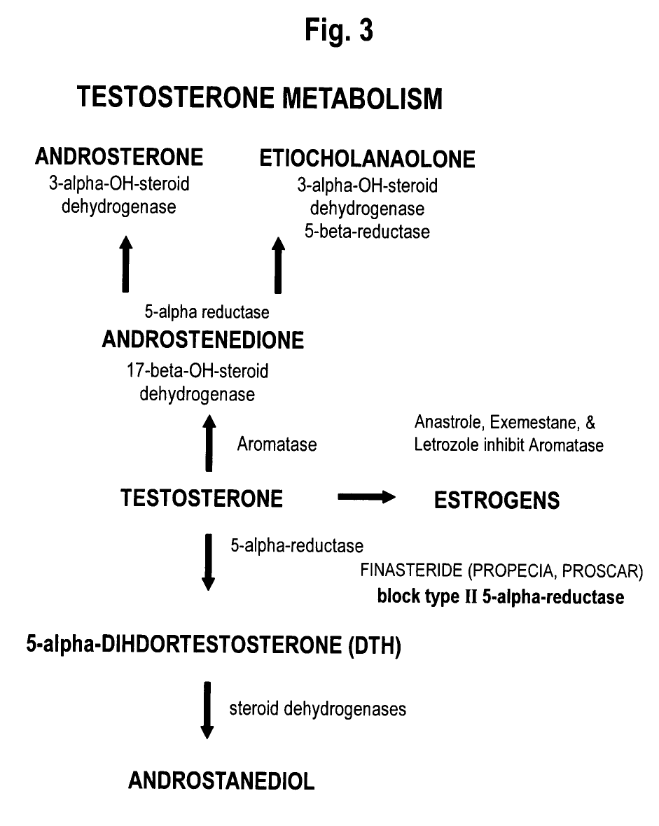

The present inventors have found that in many subjects who have been determined to have a high level of mercury (and thus suffer from mercury toxicity) also have low levels of plasma reduced glutathione (Methods for determining the levels of plasma reduced glutathione are well known to those skilled in the art). Glutathione is known to play a role in the testosterone pathway. The testosterone pathway is a part of the steroidogenic pathway (See FIG. 1). More specifically, glutathione functions as a cofactor with hydroxysteroid transferase (HST) in converting DHEA to DHEA-S (See FIG. 2). Most DHEA that is produced in the testosterone synthesis pathway is stored as DHEA-S, thereby reducing the amount that is made into androstrenediol and then eventually into testosterone (See FIGS. 2 and 3).

While not wishing to be bound by any theory, the present inventors believe that when the levels of glutathione in a subject suffering from high levels of mercury are low, HST is inhibited or its level is reduced in its function in converting DHEA to DHEA-S. The result is that the pathway shifts and the amount of testosterone produced in the subject increases. In fact, subjects having a high level of mercury, frequently are determined to have high levels of total serum testosterone. As the level of total serum testosterone in the subject increases, the more testosterone is available to bind with mercury. When testosterone binds with mercury a complex is formed. These testosterone-mercury chloride complexes are difficult to remove from the subject with a chelating agent.

The present inventors have found that the level of mercury in a subject can be lowered by treating a subject with a combination of a pharmaceutically effective amount of at least one luteinizing hormone releasing hormone composition and a pharmaceutically effective amount of at least one chelating agent and then by repeating this treatment until the level of mercury in said subjects has been lowered. The at least one luteinizing hormone binds to the LHRH receptor and thus prevents the production of gonadotropins, such as LH and FSH. LH and FSH stimulate the gonads. More specifically, in the testes, LH binds to receptors on Leydig cells, stimulating synthesis and secretion of testosterone. Theca cells in the ovary respond to LH stimulation by secretion of testosterone, which is converted into estrogen by adjacent granulosa cells. By reducing the amount of testosterone being produced in a subject determined to have high levels of mercury, less testosterone is available to bind to mercury. Because few testosterone-mercury complexes are formed, more mercury can be removed by administering to the subject at least one chelating agent.

The above-described methods (i.e., treatment regimen) are repeated as long as necessary until the level of mercury and total serum testosterone in the subject is reduced or lowered. Preferably, the level of mercury in the subject is lowered or reduced to a level that is undetectable (using any of the hereinbefore described medical tests) and the total serum testosterone is lowered or reduced to a level that is well within the normal range for the patient's age and sex and that these reduced levels of mercury and total serum testosterone remain lowered or reduced for a period of at least three months. A determination that the levels of mercury and total serum testosterone in a subject has been reduced or lowered can be made by using any medical test, such as a whole blood test or urine test, as described previously herein. The medical test can be performed as many times as necessary in order to determine whether or not the levels of mercury and total serum testosterone in the subject have been lowered.

The at least one antiandrogenic hormone is optionally administered to a subject. This treatment is administered to a subject because as the testosterone-mercury complexes begin to break apart, there is the potential to release biologically active testosterone into the body. The result is that the released biologically active testosterone may interact at the cellular level with deposit of mercury within cells, and thus produce testosterone-mercury toxicity to such cells. The at least one antiandrogenic hormone administered to a subject can help minimize the functioning of released biologically active testosterone, and hence minimize the potential for testosterone-mercury toxicity to cells within the subject.

The above method can not only be used to treat subject having a high level of mercury (and who suffers from mercury toxicity), but can also be used to treat diseases and disorders that have a mercury component. Such diseases and disorders include, but are not limited to, autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, migraine, toxic encephalopathy, cerebral degenerations, anterior horn cell disease, spinocerebellar disease, extrapyramidal disease and myopathy. The present invention also contemplates that subjects having a high level of mercury may also have one or more of the aforementioned diseases or disorders.

In another embodiment, the inventors of the present invention have found that children who have been diagnosed with autism and who also suffer from a high level of mercury (i.e. mercury toxicity) particularly benefit from the methods of the present invention as described herein. Autistic children, male or female, between the ages of two (2) and seventeen (17), particularly benefit from the methods of the present invention. Autistic children who are determined to have a high level of mercury, using any of the hereinbefore described medical tests known to those skilled in the art, can be treated pursuant to the treatment regimen described previously herein. The methods of the present invention have also been found to be useful in treating autistic children who have a high level of mercury and who have also been diagnosed with precocious puberty.

The effectiveness of the above-identified methods in treating children suffering from autism can be monitored or demonstrated though the use of ATEC (Autism, Treatment, Evaluation, Checklist) Form that was developed by the Autism Research Institute (San Diego, Calif.). The ATEC is a one-page form developed by Bernard Rimland and Stephen M. Edelson. It consists of 4 subtests:
1. Speech/Language/Communication (14 items—scores can range from 0-28).
2. Sociability (20 items—scores can range from 0-40).
3. Sensory/Cognitive Awareness (18 items—scores can range from 0-36).
4. Health/Physical/Behavior (25 items—scores can range from 0-75).
   The Autism Research Institute calculates four subscale scores and a total score (total scores can range from 0-180) from the ATEC form. The scores are weighted according to the response and the corresponding subscale. The higher the subscale and total score, the more impaired the subject. The lower the subscale and total score, the less impaired the subject.

In yet another embodiment, the present invention relates to a method of assessing the risk of whether a child is susceptible of developing autism. More specifically, the inventors have found that children, particularly male children, who have a high level of serum testosterone have a greater risk of developing autism, particularly if these children are exposed to mercury, such as through food, vaccines containing mercury as a preservative, environmental pollution, etc. Therefore, the present invention also a physician to determine, based on a child's total serum testosterone level, whether a child would be at risk of developing autism if that child were exposed to mercury.

The method involves first determining the total serum testosterone level of a child, male or female, between the ages of eight (8) months old to eighteen (18) years old by obtaining a test sample from said child. Methods for determining the level of total serum testosterone from a test sample are well known in the art. Once the total serum testosterone level of that child has been determined, that level is compared against the reference level for a child of the same age and gender. Reference levels tend to vary depending on the laboratory performing the test. If the child's total serum testosterone level is at the reference level or greater than the reference level for total serum testosterone, then the child is considered to be at risk for developing autism if that child were to be exposed to mercury. Therefore, using this information, a physician could weigh the benefits and risks associated with giving a child with a high total serum testosterone level one or more vaccinations that contains mercury as a preservative. In contrast, if the child's total serum testosterone level is not at the reference level or greater than the reference level for total serum testosterone, then such a child would not be considered to be at risk for developing autism if that child were to be exposed to mercury. For example, at age 8 months, the total serum testosterone level of a male baby is determined to be 8 ng/dL. The reference level of total serum testosterone for a male baby at a similar age at the laboratory is from 1-10 ng/dL. Therefore, given that the child's total serum testosterone level is not at or above the reference level, a determination would be made that this child would be at a low risk of developing autism if exposed to mercury. By way of another example, at age 1 year, the total serum testosterone level of a female baby is determined to be 10 ng/dL. The reference level of total serum testosterone for a female baby at a similar age at the laboratory is from 1-10 ng/dL. Therefore, given that this child's total serum testosterone level is at the reference level, a determination would be made that this child would be at a high risk of developing autism if exposed to mercury. By way of yet another example, at age 18 months, the total serum testosterone level of a male baby is determined to be 18 ng/dL. The reference level of total serum testosterone for a male baby at a similar age at the laboratory is from 1-20 ng/dL. Therefore, given that this child's total serum testosterone level is below the reference level, a determination would be made that this child would not be at risk of developing autism if exposed to mercury. By way of yet another example, at age 2 years, the total serum testosterone level of a male baby is determined to be 27 ng/dL. The reference level of total serum testosterone for a male baby at a similar age at the laboratory is from 1-25 ng/dL. Therefore, given that this child's total serum testosterone level is above the reference level, a determination would be made that this child would be at risk of developing autism if exposed to mercury.

Now by way of example, and not of limitation, examples of the present invention shall now be given.

### EXAMPLE 1

### Autistic Child X

The patient was an eight year old white male who was born in 1996 and diagnosed with autism (said child is hereinafter referred to as “Child X”). Child X was the product of a full term spontaneous vaginal delivery. Child X had good APGAR (Activity, Pulse, Grimace (reflex irritability), Appearance, Respiration) scores and was believed to be totally normal at birth. Child X developed normally meeting all of his developmental milestones during his first year of life. In addition, Child X had all of his childhood vaccines in keeping with the recommended childhood vaccine schedule. Specifically, at 28 weeks gestation the mother of Child X was administered a Rho immune globulin with approximately 70 micrograms of mercury. Moreover, from birth to approximately 15 months, Child X received 150 micrograms of mercury from his childhood vaccines. During his second year of life, Child X lost his language skills and declined into a fully autistic state. More specifically, Child X developed severe gastrointestinal problems that are often seen in autistic children. In fact, Child X never passed a normally formed stool. Child X's disorder fit into what is now commonly labeled as “regressive autism”.

From Oct. 21, 2000 through Feb. 3, 2002, Child X was treated with dimercaptosuccinic acid (DMSA) and spilled toxic levels of mercury in his urine. During this time, Child X was able to pedal his tricycle, his focus and attention was better and he attempted to say words. He was sleeping well and his bowel habits were better. In addition, his appetite was good and he was interacting more and exhibiting more outward expression. In fact, Child X began using scissors.

On Nov. 5, 2000, Child X's urine was collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child X's creatinine was 43.4 mg/dL. The results for the toxic metals found in Child X's urine are shown below in Table 1.

TABLE 1

| | Results | |
|---|---|---|
| Toxic Metals | μg/g creatinine | Reference Range |
| Aluminum | 26 | <35 |
| Antimony | 0.4 | <5 |
| Arsenic | 98 | <100 |
| Beryllium | <dl* | <0.5 |
| Bismuth | 0.8 | <30 |
| Cadmium | 1.4 | <2 |
| Lead | 12 | <15 |
| Mercury | 15 | <3 |
| Nickel | 21 | <12 |
| Platinum | 0.2 | <2 |
| Thallium | 0.2 | <14 |
| Thorium | <dl | <12 |
| Tin | 5.5 | <6 |
| Tungsten | <dl | <23 |
| Uranium | <dl | <1 |

*<dl = less than detection limit

As shown in Table 1 above, the level of mercury in Child X's urine was elevated.

On Sep. 30, 2001, Child X's urine was collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child X's creatinine was 54 mg/dL. The results for the toxic metals found in Child X's urine are shown below in Table 2.

TABLE 2

| | Results | |
|---|---|---|
| Toxic Metals | μg/g creatinine | Reference Range |
| Aluminum | 14 | <35 |
| Antimony | <dl* | <5 |
| Arsenic | 37 | <100 |
| Beryllium | <dl | <0.5 |
| Bismuth | <dl | <30 |
| Cadmium | 0.5 | <2 |
| Lead | 8.6 | <15 |
| Mercury | 5.4 | <3 |
| Nickel | 11 | <12 |
| Platinum | <dl | <2 |
| Thallium | 0.2 | <14 |
| Thorium | <dl | <12 |
| Tin | 1.6 | <6 |
| Tungsten | 0.3 | <23 |
| Uranium | <dl | <1 |

*<dl = less than detection limit

As shown in Table 2 above, the level of mercury in Child X's urine was elevated. As a result of this test a diagnosis of heavy metal toxicity, specifically, mercury toxicity, was made.

While Child X did exhibit some improvement as a result of the DMSA treatment, Child X continued to be in special class at school and received intensive speech and other behavior therapy. Child X was still unable to speak any words and could not even point to his own body parts. Child X followed verbal commands poorly, if at all, and still would only minimally interact with his peers. His behavior problems had become increasingly intolerable with age. For example, on one occasion, he severely bit his father.

Child X's prior medical work up had included chromosomes indicating that he was a 46, XY without consistent structural or numerical chromosome anomalies, a negative DNA screen for fragile-X, a negative DNA screen for Rett Syndrome and a negative newborn screen for genetic disorders. Screening for serum amino acid levels, thyroid function abnormalities and urine for reducing substances were all negative. Child X's family history is completely negative for autism or any other neurological disorders. In fact, both of his parents have advanced degrees.

On Oct. 23, 2004, Child X's blood was drawn and a laboratory work-up performed. Child X's total serum testosterone was determined to be 25 ng/dL. The reference level of total serum testosterone for a male child of Child X's age at this laboratory was from 0-25 ng/dL. Therefore, Child X's total serum testosterone was determined to be at the high end of the reference level. It was also noted that he exhibited clinical signs of precocious puberty including increased body hair and sexual masturbatory behavior. Therefore, a diagnosis of precious puberty was made. Child X had normal CBC, liver, and kidney function testing.

On Nov. 23, 2004, prior to the initiation of therapy in Child X, the severity of autistic symptoms in Child X were assessed using an ATEC (Autism, Treatment, Evaluation, Checklist) Form developed by the Autism Research Institute (San Diego, Calif.). It was observed that Child X overall had severe autistic symptoms placing him in the 90-99 percentile of severity, with Child X being most profoundly affected in the areas of sociability and sensory/cognitive awareness placing him in the 90-99 percentile of severity.

On Nov. 24, 2004, Child X was given a single shot of LUPRON DEPOT® (leuprolide acetate, Takeda Pharmaceutical Company Limited, Osaka, Japan) in the amount of 22.5 mg. No observable side effects were noted. Within a few days, Child X's behavior and attentiveness were noted to be markedly improved. On Dec. 1, 2004, transdermal DMPS treatment was begun on Child X. Specifically, Child X received a 1.5 mg transdermal DMPS dose/kg bodyweight every other day. After initiation of this chelation therapy, Child X was observed to become somewhat more hyperactive but this soon stabilized as he adjusted to the therapeutic regimen. On Dec. 3, 2004, Child X's total serum testosterone level was tested and determined to be 17 ng/dL. On Jan. 11, 2005, the total serum testosterone level of Child X was again tested and determined to be 19 ng/dL. Because of the improvement exhibited by Child X and the absence of observable side effects, Child X was given a second shot of LUPRON DEPOT® (22.5 mg) on Jan. 20, 2005. On Jan. 22, 2005, oral DMSA treatment was begun. Specifically, Child X received 7.5 mg DMSA/kg bodyweight three times per day of the oral DMSA every other day, on the days when he was also being administered transdermal DMPS (1.5 mg transdermal DMPS dose/kg bodyweight). On Jan. 28, 2005, the total serum testosterone level of Child X was tested and determined to be 32 ng/dL. On Feb. 10, 2005, the total serum testosterone level of Child X was again tested and determined to be 25 ng/dL. On Feb. 19, 2005, treatment was begun with cyproterone acetate (Androcur (Schering AG, Germany)). Specifically, Child X received 50 mg tablets three times per day. On Feb. 28, 2005, the total serum testosterone level of Child X was again tested and determined to be less than 10 ng/dL. On Mar. 18, 2005, the total serum testosterone level of Child X was again tested and determined to be 20 ng/dL. On Mar. 25, 2005, Child X was given a third shot of LUPRON DEPOT® (22.5 mg). On Apr. 8, 2005, the total serum testosterone level of Child X was tested and determined to be 10 ng/dL. On May 5, 2005, the total serum testosterone level of Child X was tested and determined to be less than 10 ng/dL. On May 25, 2005, Child X was given a fourth shot of LUPRON DEPOT® (22.5 mg). On Jun. 28, 2005, the total serum testosterone level of Child X was tested and determined to be 20 ng/dL. On Jul. 14, 2005, Child X was given a fifth shot of LUPRON DEPOT® (22.5 mg). On Aug. 15, 2005, the total serum testosterone level of Child X was tested and determined to be 23 ng/dL.

Child X was assessed by laboratory work-up for biochemical and genomic susceptibility factors to mercury toxicity. On Aug. 15, 2005, Child X's blood was drawn and a laboratory work-up performed. Child X's serum homocysteine was determined to be 5.0 micromoles/L. The reference level of serum homocysteine for a male child of Child X's age at this laboratory was 5.10-13.9 micromoles/L. On Aug. 18, 2005, Child X's blood was drawn again and another laboratory work-up performed. Child X's plasma cysteine was 2.72 mg/dL, plasma sulfate was 2.90 mg/dL, and plasma reduced glutathione was 20 mg/dL. The reference levels for each of these tests for a male child of Child X's age at this laboratory were 3.10-3.90 mg/dL for plasma cysteine, 2.90 mg/dL for plasma sulfate and ≧32 mg/dL for plasma reduced glutathione, respectively.

Within days of the first shot of LUPRON DEPOT® on Nov. 24, 2004, Child X's gastrointestinal symptoms were markedly improved. More specifically, Child X produced normal stools for the first time in seven years. A remarkable improvement in his behavior, attentiveness and mentation were also observed within a few days of the first LUPRON DEPOT® shot. Child X was able to point to most of his body parts accurately and he began to try to imitate speech sounds. Child X's ability to follow verbal-commands improved markedly and he began to interact with his siblings and peers. Within a few days of the second shot of LUPRON DEPOT®, Child X learned to swing by himself using leg timing for propulsion. Prior to receiving any of the shots of LUPRON DEPOT®, Child X could not even stay on the swing when pushed by others. Child X also began to be able to feed himself and his attention span and interest for the first time allowed him to watch and be interested in television shows. Child X began to play interactively with toys that he had never done previously. Child X also began, for the first time, to say “no” and to specifically ask for items that he wanted. Child X continues to improve on a daily basis in his mentation, attempts at speech, his interaction with others and his environment. Child X's bowel problems seem to be cured in that he continues to form normal stools. Child X continues to rapidly progress in his behavior and learning.

The improvement in Child X's mentation has been quantitatively documented. It was observed that Child X's Individualized Report Card I for the school year prior to initiation of therapy (namely, the 2003-04 school year) demonstrated that Child X had not mastered any skills in the areas of self help, general knowledge, language, social and emotional development, motor development and enrichment activities. Subsequently, it was observed that Child X's Individualized Report Card I for the mid reporting of the school year while receiving the above described therapy (namely, the 2004-05 school year) demonstrated that Child X had mastered skills in the areas of self help (uses eating utensils appropriately; washes hands independently; takes care of own toileting), general knowledge (observes likenesses and differences in objects and pictures; classifies objects according to color and shape; has left/right orientation), language (follows oral directions), social and emotional development (cooperates in group activities; accepts adult guidance; accepts consequences of own behavior; demonstrates adequate self-control; follows school rules; respects rights and property of others; demonstrates good manners) and motor development (traces simple lines; runs; jumps; hops; throws a ball). It was then observed that Child X's Individualized Report Card I for the end reporting of the school year while receiving the above described therapy (namely, the 2004-05 school year) demonstrated that Child X had mastered skills in the areas of self help (uses eating utensils appropriately; taking off and putting on his outer garments; washes hands independently; takes care of own toileting), general knowledge (recognizes and names body parts; recognizes name in print; writes name from memory; observes likenesses and differences in objects and pictures; classifies objects according to color, size and shape; has left/right orientation), language (states his full name; initiates greetings and farewells; responds to greetings and farewells; asks for assistance when necessary; speaks in short phrases; uses simple sentences; follows oral directions; attends to the speaker), social and emotional development (cooperates in group activities; accepts adult guidance; accepts consequences of own behavior; demonstrates adequate self-control; follows school rules; respects rights and property of others; demonstrates good manners; attempts new tasks in a positive manner) motor development (traces simple lines; traces name; copies name; runs; jumps; hops; catches a ball; throws a ball) and enrichment activities (participates in group singing; responds to rhythms and music; participates in activities; participates in food preparation activities). Additionally, on Jul. 30, 2005, an ATEC Form was used to evaluate the severity of autistic symptoms in Child X (therapy treatment day 248). It was observed that Child X had shown significant overall improvement from the previous ATEC form evaluation conducted on Nov. 23, 2004. Specifically it was observed that Child had improved on the ATEC form from the 90-99 percentile of autistic severity on Nov. 23, 2004 to the 20-29 percentile of autistic severity on Jul. 30, 2005. It was observed that Child X had shown the most significant improvements in the areas of sociability (90-99 percentile of autistic severity on Nov. 23, 2004 to the 20-29 percentile of autistic severity on Jul. 30, 2005) and sensory/cognitive awareness (90-99 percentile of autistic severity on Nov. 23, 2004 to the 20-29 percentile of autistic severity on Jul. 30, 2005).

### EXAMPLE 2

### Autistic Child Y

The patient was a six year old white male who was born in 1999 and diagnosed with autism (said child is hereinafter referred to as “Child Y”). Child Y was the product of a full term spontaneous vaginal delivery. Child Y had good APGAR scores and was believed to be totally normal at birth. Child Y developed normally meeting all of his developmental milestones during his first year of life. In addition, Child Y had all of his childhood vaccines in keeping with the recommended childhood vaccine schedule. Specifically, from birth to 18 months of age, Child Y had received 137.5 micrograms of mercury from his childhood vaccines. By the end of his second year of life, Child Y lost all of his language skills and declined into a fully autistic state. More specifically, Child Y developed severe gastrointestinal problems that are often seen in autistic children. Child Y never passed a normally formed stool. In fact, Child Y had an endoscopy on Jun. 23, 2003 which showed terminal ileal lymphonodular hyperplasia and inflammatory nodules of the rectosigmoid. The ileal pathology was confirmed on biopsy, and the remainder of the colon appeared to be normal. The upper endoscopy was impressive in that streaking nodular distal esophagitis was noted grossly and confirmed histologically. Child Y's disorder fit into what is now commonly labeled as “regressive autism”.

From Jun. 26, 2002 to May 2, 2003, Child Y was treated with DMSA and spilled toxic levels of mercury in his urine. During this time, Child Y did not show significant improvement in his autism.

On Jun. 29, 2002, Child Y's urine was collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child Y's creatinine was 6.9 mg/dL. The results for the toxic metals found in Child Y's urine are shown below in Table 3.

TABLE 3

| | Results | |
|---|---|---|
| Toxic Metals | μg/g creatinine | Reference Range |
| Aluminum | <dl* | <35 |
| Antimony | <dl | <5 |
| Arsenic | 31 | <100 |
| Beryllium | <dl | <0.5 |
| Bismuth | <dl | <30 |
| Cadmium | <dl | <2 |
| Lead | <dl | <15 |
| Mercury | 29 | <3 |
| Nickel | <dl | <12 |
| Platinum | <dl | <2 |
| Thallium | 0.9 | <14 |
| Thorium | <dl | <12 |
| Tin | 5.1 | <6 |
| Tungsten | <dl | <23 |
| Uranium | <dl | <1 |

*<dl = less than detection limit

As shown in Table 3 above, the level of mercury in Child Y's urine was elevated. On Jul. 15, 2002, a diagnosis of heavy metal toxicity, specifically, mercury toxicity, was made.

On Dec. 24, 2002, Child Y's urine was again collected and examined for toxic metals, specifically, aluminum, antimony, arsenic, beryllium, bismuth, cadmium, lead, mercury, nickel, platinum, thallium, thorium, tin, tungsten and uranium. The toxic metals were reported as μg/g creatinine in order to account for urine dilution variations. Child Y's creatinine was 8.6 mg/dL. The results for the toxic metals found in Child Y's urine are shown below in Table 4.

TABLE 4

| | Results | |
|---|---|---|
| Toxic Metals | μg/g creatinine | Reference Range |
| Aluminum | <dl* | <35 |
| Antimony | <dl | <5 |
| Arsenic | 23 | <100 |
| Beryllium | <dl | <0.5 |
| Bismuth | <dl | <30 |
| Cadmium | 1.6 | <2 |
| Lead | <dl | <15 |
| Mercury | 11 | <3 |
| Nickel | <dl | <12 |
| Platinum | <dl | <2 |
| Thallium | 0.6 | <14 |
| Thorium | <dl | <12 |
| Tin | 3.3 | <6 |
| Tungsten | 1.6 | <23 |
| Uranium | <dl | <1 |

*<dl = less than detection limit

As shown in Table 4 above, the level of mercury in Child Y's urine was still very elevated.

Child Y was assessed by laboratory work-up for biochemical and genomic susceptibility factors to mercury toxicity. On Jan. 20, 2003, Child Y's blood was drawn and a laboratory work-up performed. Child Y's plasma cysteine was determined to be 2.58 mg/dL. The reference level of plasma cysteine for a male child of Child Y's age at this laboratory was 3.10-3.90 mg/dL. On Jun. 2, 2004, Child Y's blood was drawn and a laboratory work-up performed. Child Y's plasma reduced glutathione was determined to be 20 mg/dL. The reference level of plasma reduced glutathione for a male child of Child Y's age at this laboratory was >32 mg/dL, respectively. In addition, a genomic survey was performed on Nov. 3, 2003 on Child Y which demonstrated that Child Y had SNPs in the MTHFR gene.

On Jan. 29, 2005, Child Y's blood was drawn and a laboratory work-up performed. Child Y's total serum testosterone was determined to be 20 ng/dL. The reference level of total serum testosterone for a male child of Child Y's age at this laboratory was from 0-20 ng/dL. Therefore, Child Y's total serum testosterone was determined to be at the high end of the reference level. It was also noted that he exhibited clinical signs of precocious puberty including increased body hair and genital development. Therefore, a diagnosis of precious puberty was made. Child Y had normal CBC, liver, and kidney function testing.

On Apr. 1, 2005, prior to the initiation of therapy in Child Y, the severity of autistic symptoms in Child Y were assessed using the ATEC Form. It was observed that Child Y overall had severe autistic symptoms placing him in the 70-79 percentile of severity, with Child Y being most profoundly affected in the area of sensory/cognitive awareness placing him in the 90-99 percentile of severity.

On Apr. 2, 2005, Child Y was given a single shot of LUPRON DEPOT® (leuprolide acetate, Takeda Pharmaceutical Company Limited, Osaka, Japan) in the amount of 22.5 mg. No observable side effects were noted. Within a few days, Child Y's gastrointestinal symptoms began to improve and he began to have well formed normal stools, which he had not had previously. In addition, Child Y's behavior and attentiveness were noted to be markedly improved. In addition, improvement in Child Y's receptive and expressive language skills has been noted. Child Y is trying to say more words and is repeating more words. On Apr. 5, 2005, transdermal DMPS treatment was begun on Child Y. Specifically, Child Y received a 1.5 mg transdermal DMPS dose/kg bodyweight every other day. On Apr. 16, 2005, the total serum testosterone level of Child Y was tested and determined to be 48 ng/dL. On Apr. 30, 2005, the total serum testosterone level of Child Y was again tested and determined to be 12 ng/dL. On May 14, 2005, the total serum testosterone level of Child Y was again tested and determined to be less than 10 ng/dL. On May 21, 2005, Child Y was given a second shot of LUPRON DEPOT(& (22.5 mg). On May 22, 2005, treatment was begun with cyproterone acetate (Androcur (Schering AG, Germany)). Specifically, Child Y received 50 mg tablets three times per day. On May 23, 2005, oral DMSA treatment was begun. Specifically, Child Y received 7.5 mg DMSA/kg bodyweight three times per day of the oral DMSA every other day, on the days when he was also being administered transdermal DMPS (1.5 mg transdermal DMPS dose/kg bodyweight). On Jun. 18, 2005, the total serum testosterone level of Child Y was again tested and determined to be 17 ng/dL. On Jul. 2, 2005, the total serum testosterone level of Child Y was again tested and determined to be 16 ng/dL. On Jul. 9, 2005, Child Y was given a third shot of LUPRON DEPOT(R (22.5 mg). On Jul. 16, 2005, the total serum testosterone level of Child Y was again tested and determined to be 15 ng/dL. On Jul. 30, 2005, the total serum testosterone level of Child Y was again tested and determined to be 13 ng/dL.

It has been observed as the therapy has progressed that Child Y has begun to show skills that were not apparent prior to the initiation of therapy. Child Y has begun to visually recognize and verbally call for his mother use appropriate expressive language skills. It has also been observed that Child Y has begun to visually recognize, communicate, and interact (showing increasing levels of affection) with other members of the family.

Specifically, in quantitative terms, an ATEC form on May 30, 2005 was used to evaluate the severity of autistic symptoms in Child Y (therapy treatment day 58). It was observed that Child Y showed an overall improvement with the most significant improvement in the area of speech/language/communication (80-89 percentile of autistic severity on Apr. 1, 2005 to 60-69 percentile of autistic severity on May 30, 2005).

Subsequently, Child Y's teaching assistant has specifically documented the following newly acquired skills for Child Y observed for period from Jul. 18, 2005 through Aug. 26, 2005:
“Child Y hardly ever initiates requested actions and seldom words. However, I have found that he knows a lot. If I offer him my arm, he will use it as a pointer. This is hardly foolproof and not all results are positive as he does not always focus. If he is wandering in his mind, his choices are not correct. If I tell him to focus and he does, his use of my arm feels much more purposeful and is highly accurate for the tasks currently presented to him. We began doing this the end of July. In the interim, it has become clear that he knows all the letters of the alphabet, both upper case and lower case. He also can read digits at least to 100. He can read many words. We spread flash cards with words in front of him and asked him to “show me”: ‘horse’ for example. He has seen these cards many times in the past. He can match animals to their pictures and color cubes to color mats. Also shapes. I have just begun using my hand under Child Y's to see if he would write. This is harder as his touch is light and I have to hold the pen. Nevertheless, I am sure that Child Y can both recognize and spell his name. When I ask him to write his name, I must tell him his first and last name. He goes right to it and has completed it four times. The third day I also asked him to write one plus one equals two and followed with a one for the next problem. He then said ‘+2=3’. I asked him after that to do the next one without specifying what to write and he wrote ‘1+3=4’. We did not use equations, but instead wrote in columns. Today, I asked him to write a three word sentence and he wrote it. I did not spell any of the words for him. (Please note that I am not at all sure that this is free from my influence, but I am sure that Child Y knows a great deal and can read quite well.)
He is beginning to take charge of the writing when we write with my hand over his and he is holding the pen. We have been writing this way for several months. With respect to words: Child Y seems to be using words a little more readily recently. Also, if I ask him what a pig says, he may provide an incorrect ‘sound’, but it will be an animal sound. If I ask him for a color, he may provide an incorrect color, but it will be a color. Child Y also seems to be able to recognize clocks showing the hour and the half hour for any ‘hour’. He knows how to read a digital time. He also can respond correctly to ‘Show me: quarter, dime, nickel, penny’ (Show me commands require his use of my hand).”

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. A method of lowering the level of mercury in a subject suffering from mercury toxicity, the method comprising the steps of:
a) administering to said subject a pharmaceutically effective amount of at least one luteinizing hormone releasing hormone composition;
b) administering to said subject a pharmaceutically effective amount of at least one chelating agent; and
c) repeating step a), step b) or steps a) and b) as necessary to lower the level of mercury in said subject.

2. The method of claim 1 wherein the at least one luteinizing hormone composition is a luteinizing hormone releasing hormone (“LHRH”) analogue, a LHRH agonist, a LHRH antagonist or combinations thereof.

3. The method of claim 2 wherein the at least one luteinizing hormone composition is a LHRE agonist.

4. The method of claim 3 wherein the LHRH agonist is leuprolide acetate.

5. The method of claim 1 further comprising the step of administering to the subject a pharmaceutically effective amount of at least one antiandrogenic hormone either prior to or after step c).

6. The method of claim 5 wherein the at least one antiandrogenic hormone is cyproterone acetate, finasteride, bicalutamide, novaldex, nilandron, flutamide, progesterone or combinations thereof.

7. The method of claim 1 wherein the subject is a human male or a human female.

8. The method of claim 7 wherein the human male or human female is suffering from a disorder selected from the group consisting of: autism, autism spectrum disorders, attention deficit disorder, attention deficit hyperactivity disorder, mental retardation, Asperger's syndrome, childhood psychoses, stammering, stuttering, tics, repetitive movements, eating disorders, sleep disorders, enuresis, developmental language disorders, developmental speech disorders, developmental delay, Alzheimer's disease, diabetes, heart disease, obesity, amyotrophic lateral sclerosis, nephritic syndrome, renal failure, asthma, systemic lupus, autoimmune thyroiditis, rheumatoid arthritis, arthritis, vasculities, myelitis, glomerulonephritis, optic neuritis, infantile cerebral palsy, epilepsy, migraine, toxic encephalopathy, cerebral degenerations, anterior horn cell disease, spinocerebellar disease, extrapyramidal disease and myopathy.

9. The method of claim 7 wherein the subject is a human male or human female is a child having an age between 2 years and 17 years.

10. The method of claim 9 wherein the human male or human female child has autism.

11. The method of claim 10 wherein the child is a male child.

12. The method of claim 11 wherein the human male child has been diagnosed with precocious puberty.

13. The method of claim 1 wherein the chelating agent is administered orally, transdermally, intravenously, orally and transdermally, orally, transdermally and intravenously, orally and intravenously or transdermally and intravenously.

14. A method of lowering the level of mercury in a human child suffering from mercury toxicity, wherein said child also suffers from autism, the method comprising the steps of:
a) administering to said child a pharmaceutically effective amount of at least one luteinizing hormone releasing hormone composition;
b) administering to said child a pharmaceutically effective amount of at least one chelating agent; and
c) repeating step a), step b) or steps a) and b) as necessary to lower the level of mercury in said subject.

15. The method of claim 14 wherein said child has an age of from about 2 years old to about 17 years old.

16. The method of claim 14 wherein the at least one luteinizing hormone composition is a luteinizing hormone releasing hormone (“LHRH”) analogue, a LHRH agonist, a LHRH antagonist or combinations thereof.

17. The method of claim 16 wherein the at least one luteinizing hormone composition is a LHRH agonist.

18. The method of claim 17 wherein the LHR-H agonist is leuprolide acetate.

19. The method of claim 14 further comprising the step of administering to the subject a pharmaceutically effective amount of at least one antiandrogenic hormone either prior to or after step c).

20. The method of claim 19 wherein the at least one antiandrogenic hormone is cyproterone acetate, finasteride, bicalutamide, novaldex, nilandron, flutamide, progesterone or combinations thereof.

21. The method of claim 14 wherein the child is a male child.

22. The method of claim 14 wherein the child has been diagnosed with precocious puberty.

23. The method of claim 14 wherein the chelating agent is administered orally, transdermally, intravenously, orally and transdermally, orally, transdermally and intravenously, orally and intravenously or transdermally and intravenously.

24. A method of treating a child diagnosed with autism, wherein said child is also diagnosed with mercury toxicity, said method comprising the steps of:
a) administering to said child a pharmaceutically effective amount of at least one luteinizing hormone releasing hormone composition;
b) administering to said child a pharmaceutically effective amount of at least one chelating agent; and
c) repeating step a), step b) or steps a) and b) as necessary to treat said child.

25. The method of claim 24 wherein said child has an age of from about 2 years old to about 17 years old.

26. The method of claim 24 wherein the at least one luteinizing hormone composition is a luteinizing hormone releasing hormone (“LHRH”) analogue, a LHRH agonist, a LHRH antagonist or combinations thereof.

27. The method of claim 24 wherein the at least one luteinizing hormone composition is a LHRH agonist.

28. The method of claim 27 wherein the LHRH agonist is leuprolide acetate.

29. The method of claim 24 further comprising the step of administering to the subject a pharmaceutically effective amount of at least one antiandrogenic hormone either prior to or after step c).

30. The method of claim 29 wherein the at least one antiandrogenic hormone is cyproterone acetate, finasteride, bicalutamide, novaldex, nilandron, flutamide, progesterone or combinations thereof.

31. The method of claim 24 wherein the child is a male child.

32. The method of claim 24 wherein the child has been diagnosed with precocious puberty.

33. The method of claim 24 wherein the chelating agent is administered orally, transdermally, intravenously, orally and transdermally, orally, transdermally and intravenously, orally and intravenously or transdermally and intravenously.

34. A method of assessing the risk of whether a child is susceptible of developing autism, the method comprising the steps of:
a) determining the level of total serum testosterone from a test sample obtained from a child; and
b) assessing, based on a comparison of the level of total serum testosterone in said test sample with a reference level of total serum testosterone, whether said child is at risk of developing autism.

35. The method according to claim 34 wherein the test sample is a whole blood sample or a plasma sample.

36. The method according to claim 34 wherein a child is at risk of developing autism when said child has a total serum testosterone level that is at the reference level or greater than the reference level for total serum testosterone for a child of approximately the same age.

37. The method according to claim 34, wherein a child is not at risk of developing autism when said child has a total serum testosterone level that is lower than the reference level for total serum testosterone for a child of approximately the same age.
